(19) United States
(12) Patent Application Publication
(10) Pub. No.: US 2007/0048747 A1
(43) Pub. Date: Mar. 1, 2007

(54) Methods for assaying analytes

(76) Inventors: Thomas M. Leslie, Horseheads, NY (US); Guangshan Li, Corning, NY (US); Jeffrey G. Lynn, Tioga, PA (US); Jinlin Peng, Painted Post, NY (US)

Correspondence Address: CORNING INCORPORATED, SP-TI-3-1, CORNING, NY, 14831, US

(21) Appl. No.: 11/219,049

(22) Filed: Sep. 1, 2005

(51) Int. Cl. C12Q 1/68; G01N 33/53; C12M 3/00; G01N 33/50

(52) U.S. Cl. 435/6; 435/7.1; 435/287.2; 977/902; 436/86

(57) ABSTRACT

Described herein are methods for assaying analytes.

### BACKGROUND

There is an increasing need to be able to assay large numbers of analytes with greater sensitivity. For example, gene expression analysis using microarrays has become an important component in many stages of biological research in recent years (DeRisi, et al. 1997; Gill, et al. 2002; Hegde, et al. 2000; Monni, et al. 2001). Microarrays can simultaneously interrogate thousands of biological molecules such as genes, which generate gene expression profiles to produce a wealth of information. Such profiles are exquisitely detailed patterns and are characteristic of the response of cells to their environments, differentiation into specialized tissues, or treatments. There are three segments for microarray technologies: 1) microarray fabrication; 2) experimental design and performance; and 3) microarray data mining. Although significant progress has been made over the last decade, there are still many challenges among each of the segments (Jenssen, et al. 2002; Kepler, et al. 2002; Li, et al. 2003; Pan, et al. 2002; Relógio, et al. 2002; Yue, et al. 2001). One such challenge is to develop a slide surface for covalent immobilization of short oligonucleotides (oligo) and an assay kit to generate highly consistent data with low cost.

In the case of oligonucleotides, the assay sensitivity for short oligo microarrays is much lower than that of long oligo and cDNA microarrays because the stability of a short hybrid is much weaker than that of a long hybrid. One way to improve the sensitivity is to achieve a stable and oriented immobilization of short oligos on a surface by covalent chemistries through amine modified terminals. There are several kinds of surface chemistries that have been utilized for covalent immobilization of molecules. Three of them are frequently used in microarray applications. Aldehyde surfaces such as SuperAldehyde slide (Telechem Inc.) can allow biomolecules with primary amine groups to be attached covalently by undergoing a Schiff base reaction. Epoxide groups can react with amines to form a covalent linkage. Slides with epoxide surface chemistry are now widely available. Another choice for covalent attachment is the N-hydroxysuccinimide (NHS) active ester such as 3D code-link slides from SurModics Inc. Biomolecules containing primary amine groups can selectively immobilize biomolecules on the surface through an amide bond.

Another approach is to have anhydride groups present on the surface of the substrate that can react with a biomolecule of interest. For example, poly(styrene-co-maleic anhydride) (SMA) is a copolymer containing reactive anhydride groups, which can quickly react with primary and secondary amines. SMA can be efficiently coated on the amine modified glass surface through covalent bonding between anhydride and amino groups and provide stable SMA coating. However, environmental contaminants can also easily bind to the SMA surface and cause uneven, high background noise, which is detrimental to microarray applications.

Described herein are methods for dramatically reducing the non-specific binding of biomolecules on a substrate. High specificity and sensitivity are also achieved using the methods described herein. The methods described herein offer time saving, economical, consistent, and efficient analysis of an analyte. For example, the methods described herein permit the measurement of biomolecule abundance and/or molecule interaction in biological samples.

### SUMMARY

Described herein are methods for assaying analytes. The advantages of the materials, methods, and articles described herein will be set forth in part in the description which follows, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying drawings, which are incorporated in and constitute a part of this specification, illustrate several aspects described below. It will be appreciated that these drawings depict only typical embodiments of the materials, articles, and methods described herein and are therefore not to be considered limiting of their scope.

FIG. 1 shows hybridization images using different blockers.

FIG. 2 shows five different hybridization buffers were evaluated by hybridization, where the SMA slides were blocked with either DMEDA or AEE before hybridization.

FIG. 3 shows the effect of different PVP molecular weights on SMA hybridization performance, where the slides were blocked with AEE.

FIG. 4 shows the comparison of different low molecular weight polymers for hybridization on SMA.

FIG. 5 shows different combinations of polymers that were used as hybridization buffers.

FIG. 6 shows the salt effect on SMA hybridization.

FIG. 7 shows the influence humidity imparts to hybridization signal on a SMA slide.

FIG. 8 shows SMA hybridization images before and after optimizations. Panel A shows array hybridization images before optimization and panel B shows array hybridization images after optimization.

### DETAILED DESCRIPTION

Before the present materials, articles, and/or methods are disclosed and described, it is to be understood that the aspects described below are not limited to specific compounds, synthetic methods, or uses as such may, of course, vary. It is also to be understood that the terminology used herein is for the purpose of describing particular aspects only and is not intended to be limiting.

In this specification and in the claims that follow, reference will be made to a number of terms that shall be defined to have the following meanings:

Throughout this specification, unless the context requires otherwise, the word “comprise,” or variations such as “comprises” or “comprising,” will be understood to imply the inclusion of a stated integer or step or group of integers or steps but not the exclusion of any other integer or step or group of integers or steps.

It must be noted that, as used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed, then “less than or equal to 10” as well as “greater than or equal to 10” is also disclosed. It is also understood that throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

By “contacting” is meant an instance of exposure by close physical contact of at least one substance to another substance.

The term “attached” as used herein is any chemical interaction between two components or compounds. The type of chemical interaction that can be formed will vary depending upon the starting materials that are selected and reaction conditions. Examples of attachments described herein include, but are not limited to, covalent, electrostatic, ionic, hydrogen, or hydrophobic bonding.

Disclosed are compounds, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a number of different polymers and biomolecules are disclosed and discussed, each and every combination and permutation of the polymer and biomolecule are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this disclosure including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

I. Supports

a. Substrates

Described herein are supports useful for performing assays. In one aspect, the support comprises a substrate, a first tie layer attached to the substrate, a first polymer attached to the first tie layer, wherein the biomolecule attaches to the first polymer.

In a further aspect, the tie layer is covalently or electrostatically attached to the outer surface of the substrate. The term “outer surface” with respect to the substrate is the region of the substrate that is exposed and can be subjected to manipulation. For example, any surface on the substrate that can come into contact with a solvent or reagent upon contact is considered the outer surface of the substrate. The substrates that can be used herein include, but are not limited to, a microplate, a slide, or any other material that can support cell growth. In one aspect, when the substrate is a microplate, the number of wells and well volume will vary depending upon the scale and scope of the analysis. Other examples of substrates useful herein include, but are not limited to, a cell culture surface such as a 384-well microplate, a 96-well microplate, 24-well dish, 8-well dish, 10 cm dish, or a T75 flask.

For optical or electrical detection applications, the substrate can be transparent, impermeable, or reflecting, as well as electrically conducting, semiconducting, or insulating. For biological applications, the substrate material may be either porous or nonporous and may be selected from either organic or inorganic materials.

In a further aspect, the substrate comprises a plastic, a polymeric or co-polymeric substance, a ceramic, a glass, a metal, a crystalline material, a noble or semi-noble metal, a metallic or non-metallic oxide, a transition metal, or any combination thereof. Additionally, the substrate can be configured so that it can be placed in any detection device. In one aspect, sensors can be integrated into the bottom/underside of the substrate and used for subsequent detection. These sensors could include, but are not limited to, optical gratings, prisms, electrodes, and quartz crystal microbalances. Detection methods could include fluorescence, phosphorescence, chemiluminescence, refractive index, mass, electrochemical. In one aspect, the substrate is a resonant waveguide grating sensor.

In a further aspect, the substrate can be composed of an inorganic material. Examples of inorganic substrate materials include, but are not limited to, metals, semiconductor materials, glass, and ceramic materials. Examples of metals that can be used as substrate materials include, but are not limited to, gold, platinum, nickel, palladium, aluminum, chromium, steel, and gallium arsenide. Semiconductor materials used for the substrate material include, but are not limited to, silicon and germanium. Glass and ceramic materials used for the substrate material can include, but are not limited to, quartz, glass, porcelain, alkaline earth aluminoborosilicate glass and other mixed oxides. Further examples of inorganic substrate materials include graphite, zinc selenide, mica, silica, lithium niobate, and inorganic single crystal materials.

In a further aspect, the substrate comprises a porous, inorganic layer. Any of the porous substrates and methods of making such substrates disclosed in U.S. Pat. No. 6,750,023, which is incorporated by reference in its entirety, can be used herein. In one aspect, the inorganic layer on the substrate comprises a glass or metal oxide. In another aspect, the inorganic layer comprises a silicate, an aluminosilicate, a boroaluminosilicate, a borosilicate glass, or a combination thereof. In a further aspect, the inorganic layer comprises TiO₂, SiO₂, Al₂O₃, Cr₂O₃, CuO, ZnO, Ta₂O₅, Nb₂O₅, ZnO₂, or a combination thereof.

In a further aspect, the substrate can be composed of an organic material. Organic materials useful herein can be made from polymeric materials due to their dimensional stability and resistance to solvents. Examples of organic substrate materials include, but are not limited to, polyesters, such as polyethylene terephthalate and polybutylene terephthalate; polyvinylchloride; polyvinylidene fluoride; polytetrafluoroethylene; polycarbonate; polyamide; poly(meth)acrylate; polystyrene, polyethylene; or ethylene/vinyl acetate copolymer.

In a further aspect, the support can be composed of a material that possesses groups capable of attaching one or more biomolecules. For example, the substrate can be composed of one or more first polymers described herein and molded into any desired shape. In this aspect, the biomolecule and other components can be attached directly to the substrate without the use of a tie layer.

b. Tie Layer

In various aspects, the substrates described herein have a tie layer covalently bonded to the substrate; however, it is also contemplated that a different tie layer compound can be attached to the substrate by other means in combination with a tie layer compound that is covalently bonded to the substrate. For example, one tie layer compound can be covalently bonded to the substrate and a second tie layer compound can be electrostatically bonded to the substrate. In a further aspect, when the tie layer is electrostatically bonded to the substrate, the compound used to make the tie layer is positively charged and the outer surface of the substrate is treated such that a net negative charge exists so that tie layer compound and the outer surface of the substrate form an electrostatic bond.

In a further aspect, the outer surface of the substrate can be derivatized so that there are groups capable of forming a covalent bond with the tie layer compound. The tie layer can be directly or indirectly covalently bonded to the substrate. In the case when the tie layer is indirectly bonded to the substrate, a linker possessing groups that can covalently attach to both the substrate and the tie layer compound can be used. Examples of linkers include, but are not limited to, an ether group, a polyether group, a polyamine, or a polythioether. If a linker is not used, and the tie layer compound is covalently bonded to the substrate, this is referred to as direct covalent attachment.

In a further aspect, the tie layer is derived from a compound comprising one or more reactive functional groups that can react with the first polymer. The phrase “derived from” with respect to the tie layer is defined herein as the resulting residue or fragment of the tie layer compound when it is attached to the substrate. The functional groups permit the attachment of the first polymer to the tie layer. In a further aspect, the functional groups of the tie layer compound comprises an amino group, a thiol group, a hydroxyl group, a carboxyl group, an acrylic acid, an organic and inorganic acid, an ester, an anhydride, an aldehyde, an epoxide, their derivatives or salts thereof, or a combination thereof. In a further aspect, the tie layer is derived from a straight or branched-chain aminosilane, aminoalkoxysilane, aminoalkylsilane, aminoarylsilane, aminoaryloxysilane, or a derivative or salt thereof. In a further aspect, the tie layer is derived from 3-aminopropyl trimethoxysilane, N-(beta-aminoethyl)-3-aminopropyl trimethoxysilane, N-(beta-aminoethyl)-3-aminopropyl triethoxysilane, N′-(beta-aminoethyl)-3-aminopropyl methoxysilane, or aminopropylsilsesquixoxane.

The tie layer can be attached to any of the substrates described herein using techniques known in the art. For example, the substrate can be dipped in a solution of the tie compound. In a further aspect, the tie compound can be sprayed, vapor deposited, screen printed, or robotically pin printed or stamped on the substrate. This could be done either on a fully assembled substrate or on a bottom insert (e.g., prior to attachment of the bottom insert to a holey plate to form a microplate).

C. First Polymer

In various aspects, a first polymer comprising one or more functional groups that can bind a biomolecule to the substrate is attached to the tie layer. The “functional group” on the first polymer or any polymer described herein permits the attachment of the first polymer to the first tie layer or the biomolecule. The first polymer or subsequent polymers can have one or more different functional groups. It is also contemplated that some first polymer may also be attached to the outer surface of the substrate as well as attached to the first tie layer. Alternatively, the first polymer may be in contact with the outer surface of the substrate and still be attached to the first tie layer. In a further aspect, the first polymer can be covalently and/or electrostatically attached to the first tie layer. It is also contemplated that two or more different first polymers can be attached to the first tie layer.

The first polymer can be water-soluble or water-insoluble depending upon the technique used to attach the first polymer to the first tie layer. The first polymer can be either linear or non-linear. For example, when the first polymer is non-linear, the first polymer is a dendritic polymer. The first polymer can be a homopolymer or a copolymer.

In a further aspect, the first polymer comprises at least one electrophilic group susceptible to nucleophilic attack. Not wishing to be bound by theory, when the first tie layer possesses a nucleophilic group that reacts with the electrophilic group of the polymer to form a covalent bond, a negative charge is produced at the first polymer. The negative charge at the first polymer layer can then facilitate the formation of an electrostatic bond between the first polymer and a biomolecule. Alternatively, one or more electrophilic groups present on the first polymer layer can form a covalent bond with a biomolecule. In the case when a biomolecule is attached to the first polymer, the presence of specific side chains in the polymer (e.g. ethylene glycol) can help prevent non-specific binding of the biomolecule to the first polymer.

In a further aspect, the first polymer comprises at least one amine-reactive group. The term “amine-reactive group” is any group that is capable of reacting with an amine group to form a new covalent bond. The amine can be a primary, secondary, or tertiary amine. In a further aspect, the amine-reactive group comprises an ester group, an epoxide group, or an aldehyde group. In a further aspect, the amine-reactive group is an anhydride group.

In a further aspect, the first polymer comprises a copolymer derived from maleic anhydride and a first monomer. In this aspect, the amount of maleic anhydride in the first polymer is from 5% to 50%, 5% to 45%, 5% to 40%, 5% to 35%, 5% to 30%, 5% to 25%, 10% to 50%, 15% to 50%, 20% to 50%, 25% to 50%, or 30% to 50% by stoichiometry (i.e., molar amount) of the first monomer. In one aspect, the first monomer selected improves the stability of the maleic anhydride group in the first polymer. In another aspect, the first monomer reduces nonspecific binding of the biomolecule to the substrate. In a further aspect, the amount of maleic anhydride in the first polymer is about 50% by stoichiometry of the first monomer. In another aspect, the first monomer comprises styrene, tetradecene, octadecene, methyl vinyl ether, triethylene glycol methyl vinyl ether, butylvinyl ether, divinylbenzene, ethylene, acrylamide, dimethylacrylamide, pyrolidone, a polymerizable oligo(ethylene glycol) or oligo(ethylene oxide), or a combination thereof.

In a further aspect, the first polymer comprises, poly(vinyl acetate-maleic anhydride), poly(styrene-co-maleic anhydride), poly(isobutylene-alt-maleic anhydride), poly(maleic anhydride-alt-1-octadecene), poly(maleic anhydride-alt-1-tetradecene), poly(maleic anhydride-alt-methyl vinyl ether), poly(triethyleneglycol methyvinyl ether-co-maleic anhydride), poly(ethylene-alt-maleic anhydride), or a combination thereof.

The amount of first polymer attached to the first tie layer can vary depending upon among other things the selection of the first tie layer, the first polymer, the biomolecule, and the analyte to be detected. In a further aspect, the first polymer comprises at least one monolayer. In a further aspect, the first polymer has a thickness of about 10 Å to about 2,000 Å. In another aspect, the thickness of the first polymer has a lower endpoint of 10 Å, 20 Å, 40 Å, 60 Å, 80 Å, 100 Å, 150 Å, 200 Å, 300 Å, 400 Å, or 500 Å and an upper endpoint of 750 Å, 1,000 Å, 1,250 Å, 1,500 Å, 1,750 Å, or 2,000 Å, where any lower endpoint can be combined with any upper endpoint to form the thickness range.

In a further aspect, the first tie layer is aminopropylsilsesquioxane and the first polymer is poly(styrene-co-maleic anhydride).

The first polymer can be attached to the substrate using techniques known in the art. For example, the substrate can be dipped in a solution of the first polymer. In another aspect, the first tie compound or first polymer can be sprayed, vapor deposited, screen printed, or robotically pin printed or stamped on the substrate. This could be done either on a fully assembled substrate or on a bottom insert (e.g., prior to attachment of the bottom insert to a holey plate to form a microplate).

d. Biomolecules

It is contemplated that one or more different biomolecules can be attached to the substrate to produce a variety of biological sensors. In a further aspect, the biomolecule can be attached covalently or electrostatically to the first polymer. The biomolecules may exhibit specific affinity for another molecule through covalent or non-covalent bonding. Examples of biomolecules useful herein include, but are not limited to, a nucleic acid molecule, an antibody, a peptide, a small molecule, a lectin, a modified polysaccharide, a synthetic composite macromolecule, a functionalized nanostructure, a synthetic polymer, a modified/blocked nucleotides/nucleoside, a modified/blocked amino acid, a fluorophore, a chromophore, a ligand, a chelate, an aptamer, a drug (e.g., a small molecule), or a hapten.

In a further aspect, the biomolecule can be a protein. For example, the protein can include peptides, fragments of proteins or peptides, membrane-bound proteins, or nuclear proteins. The protein can be of any length, and can include one or more amino acids or variants thereof. The protein(s) can be fragmented, such as by protease digestion, prior to analysis. A protein sample to be analyzed can also be subjected to fractionation or separation to reduce the complexity of the samples. Fragmentation and fractionation can also be used together in the same assay. Such fragmentation and fractionation can simplify and extend the analysis of the proteins.

In a further aspect, the biomolecule is a virus. Examples of viruses include, but are not limited to, Herpes simplex virus type-1, Herpes simplex virus type-2, Cytomegalovirus, Epstein-Barr virus, Varicella-zoster virus, Human herpesvirus 6, Human herpesvirus 7, Human herpesvirus 8, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A, Influenza virus B, Measles virus, Polyomavirus, Human Papilomavirus, Respiratory syncytial virus, Adenovirus, Coxsackie virus, Dengue virus, Mumps virus, Poliovirus, Rabies virus, Rous sarcoma virus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency cirus, Human T-cell Leukemia virus type-1, Hantavirus, Rubella virus, Simian immunodeficiency virus, Human Immunodeficiency virus type-1, Vaccinia virus, SARS virus, Human Immunodeficiency virus type-2, lentivirus, baculovirus, adeno-associated virus, or any strain or variant thereof.

In a further aspect, the biomolecule comprises a nucleic acid. The nucleic acid can be an oligonucleotide, deoxyribonucleic acid (DNA) or a fragment thereof, ribonucleic acid (RNA) or a fragment thereof, or peptide nucleic acid (PNA) or a fragment thereof. The nucleic acid can be a nucleic acid from any source, such as a nucleic acid obtained from cells in which it occurs in nature, recombinantly produced nucleic acid, or chemically synthesized nucleic acid. For example, the nucleic acid can be cDNA or genomic DNA or DNA synthesized to have the nucleotide sequence corresponding to that of naturally-occurring DNA. The nucleic acid can also be a mutated or altered form of nucleic acid (e.g., DNA that differs from a naturally occurring DNA by an alteration, deletion, substitution or addition of at least one nucleic acid residue) or nucleic acid that does not occur in nature.

In a further aspect, the nucleic acid can be present in a vector such as an expression vector (e.g., a plasmid or viral-based vector). In a further aspect, the vector is a chromosomally integrated vector. The nucleic acids useful herein can be linear or circular and can be of any size. In a further aspect, the nucleic acid can be single or double stranded DNA or RNA.

In a further aspect, the nucleic acid can be a functional nucleic acid. Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, aptamers, ribozymes, triplex forming molecules, RNAi, and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

Functional nucleic acids can be a small gene fragment that encodes dominant-acting synthetic genetic elements (SGEs), e.g., molecules that interfere with the function of genes from which they are derived (antagonists) or that are dominant constitutively active fragments (agonists) of such genes. SGEs can include, but are not limited to, polypeptides, inhibitory antisense RNA molecules, ribozymes, nucleic acid decoys, and small peptides. The small gene fragments and SGE libraries disclosed in U.S. Patent Publication No. 2003/0228601, which is incorporated by reference, can be used herein.

The functional nucleic acids of the present method can function to inhibit the function of an endogenous gene at the level of nucleic acids, e.g., by an antisense or decoy mechanism, or by encoding a polypeptide that is inhibitory through a mechanism of interference at the protein level, e.g., a dominant negative fragment of the native protein. Alternatively, certain functional nucleic acids can function to potentiate (including mimicking) the function of an endogenous gene by encoding a polypeptide which retains at least a portion of the bioactivity of the corresponding endogenous gene, and may in particular instances be constitutively active.

Functional nucleic acid molecules can interact with any macromolecule, such as DNA, RNA, polypeptides, or carbohydrate chains. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (kd) less than or equal to 10^{-6 to -12}. A representative sample of methods and techniques which aid in the design and use of antisense molecules can be found in the following non-limiting list of U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437.

Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Aptamers can bind very tightly with kds from the target molecule of less than 10⁻¹² M. It is preferred that the aptamers bind the target molecule with a kd less than 10^{-6 to -12}. Aptamers can bind the target molecule with a very high degree of specificity. For example, aptamers have been isolated that have greater than a 10000 fold difference in binding affinities between the target molecule and another molecule that differ at only a single position on the molecule (U.S. Pat. No. 5,543,293). It is preferred that the aptamer have a kd with the target molecule at least 10, 100, 1000, 10,000, or 100,000 fold lower than the kd with a background binding molecule. It is preferred when doing the comparison for a polypeptide for example, that the background molecule be a different polypeptide. Representative examples of how to make and use aptamers to bind a variety of different target molecules can be found in the following non-limiting list of U.S. Pat. Nos. 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698.

Ribozymes are nucleic acid molecules that are capable of catalyzing a chemical reaction, either intramolecularly or intermolecularly. Ribozymes are thus catalytic nucleic acids. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes (for example, but not limited to the following U.S. Pat. Nos. 5,334,711, 5,436,330, 5,616,466, 5,633,133, 5,646,020, 5,652,094, 5,712,384, 5,770,715, 5,856,463, 5,861,288, 5,891,683, 5,891,684, 5,985,621, 5,989,908, 5,998,193, 5,998,203, WO 9858058 by Ludwig and Sproat, WO 9858057 by Ludwig and Sproat, and WO 9718312 by Ludwig and Sproat) hairpin ribozymes (for example, but not limited to the following U.S. Pat. Nos. 5,631,115, 5,646,031, 5,683,902, 5,712,384, 5,856,188, 5,866,701, 5,869,339, and 6,022,962), and tetrahymena ribozymes (for example, but not limited to the following U.S. Pat. Nos. 5,595,873 and 5,652,107). There are also a number of ribozymes that are not found in natural systems, but which have been engineered to catalyze specific reactions de novo (for example, but not limited to the following U.S. Pat. Nos. 5,580,967, 5,688,670, 5,807,718, and 5,910,408). Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Representative examples of how to make and use ribozymes to catalyze a variety of different reactions can be found in the following non-limiting list of U.S. Pat. Nos. 5,646,042, 5,693,535, 5,731,295, 5,811,300, 5,837,855, 5,869,253, 5,877,021, 5,877,022, 5,972,699, 5,972,704, 5,989,906, and 6,017,756.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. It is preferred that the triplex forming molecules bind the target molecule with a kd less than 10−6, 10−8, 10−10, or 10−12. Representative examples of how to make and use triplex forming molecules to bind a variety of different target molecules can be found in the following non-limiting list of U.S. Pat. Nos. 5,176,996, 5,645,985, 5,650,316, 5,683,874, 5,693,773, 5,834,185, 5,869,246, 5,874,566, and 5,962,426.

External guide sequences (EGSs) are molecules that bind a target nucleic acid molecule forming a complex, and this complex is recognized by RNase P, which cleaves the target molecule. EGSs can be designed to specifically target an RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. (WO 92/03566 by Yale, and Forster and Altman, Science 238:407-409 (1990)).

Similarly, eukaryotic EGS/RNAse P-directed cleavage of RNA can be utilized to cleave desired targets within eukarotic cells. (Yuan et al., Proc. Natl. Acad. Sci. USA 89:8006-8010 (1992); WO 93/22434 by Yale; WO 95/24489 by Yale; Yuan and Altman, EMBO J 14:159-168 (1995), and Carrara et al., Proc. Natl. Acad. Sci. (USA) 92:2627-2631 (1995)). Representative examples of how to make and use EGS molecules to facilitate cleavage of a variety of different target molecules can be found in the following non-limiting list of U.S. Pat. Nos. 5,168,053, 5,624,824, 5,683,873, 5,728,521, 5,869,248, and 5,877,162.

It is also understood that the disclosed nucleic acids can be RNA (e.g., for RNA interference (RNAi)). It is thought that RNAi involves a two-step mechanism for RNA interference: an initiation step and an effector step. For example, in the first step, input double-stranded (ds) RNA (siRNA) is processed into small fragments, such as 21-23-nucleotide “guide sequences.” RNA amplification appears to be able to occur in whole animals. Typically then, the guide RNAs can be incorporated into a protein RNA complex which is cable of degrading RNA, the nuclease complex, which has been called the RNA-induced silencing complex (RISC). This RISC complex acts in the second effector step to destroy mRNAs that are recognized by the guide RNAs through base-pairing interactions. RNAi involves the introduction by any means of double stranded RNA into the cell which triggers events that cause the degradation of a target RNA. RNAi is a form of post-transcriptional gene silencing.

Disclosed are RNA hairpins that can act in RNAi. In one aspect, the RNAi agent can be small ribonucleic acid molecules (also referred to herein as interfering ribonucleic acids), i.e., oligoribonucleotides, that are present in duplex structures, e.g., two distinct oligoribonucleotides hybridized to each other or a single ribooligonucleotide that assumes a small hairpin formation to produce a duplex structure. By oligoribonucleotide is meant a ribonucleic acid that does not exceed about 100 nt in length, and typically does not exceed about 75 nt length, where the length in certain embodiments is less than about 70 nt. When the RNAi agent is a duplex structure of two distinct ribonucleic acids hybridized to each other, e.g., an siRNA, such as d-siRNA, the length of the duplex structure typically ranges from about 15 to 30 bp, usually from about 15 to 29 bp, where lengths between about 20 and 29 bps, e.g., 21 bp, 22 bp, can be used. Where the RNAi agent is a duplex structure of a single ribonucleic acid that is present in a hairpin formation, i.e., a shRNA, the length of the hybridized portion of the hairpin is typically the same as that provided above for the siRNA type of agent or longer by 4-8 nucleotides. The weight of the RNAi agents of this embodiment typically ranges from about 5,000 daltons to about 35,000 daltons, and in many embodiments is at least about 10,000 daltons and less than about 27,500 daltons, often less than about 25,000 daltons.

In certain aspects, instead of the RNAi agent being an interfering ribonucleic acid, e.g., an siRNA or shRNA as described above, the RNAi agent can encode an interfering ribonucleic acid, e.g., an shRNA, as described above. In other words, the RNAi agent can be a transcriptional template of the interfering ribonucleic acid. In these aspects, the transcriptional template can be a DNA that encodes the interfering ribonucleic acid.

RNAi has been shown to work in a number of cells, including mammalian cells. For work in mammalian cells it is preferred that the RNA molecules which will be used as targeting sequences within the RISC complex are shorter. For example, less than or equal to 50 or 40 or 30 or 29, 28, 27, 26, 25, 24, 23, 22, 21, 20, 19, 18, 17, 16, 15, 14, 13, 12, 11, or 10 nucleotides in length. These RNA molecules can also have overhangs on the 3′ or 5′ ends relative to the target RNA which is to be cleaved. These overhangs can be at least or less than or equal to 1, 2, 3, 4, 5, 6, 7, 8, 9, 10, 15, or 20 nucleotides long. RNAi works in mammalian stem cells, such as mouse ES cells.

For description of making and using RNAi molecules see See, e.g., Hammond et al., Nature Rev Gen 2: 110-119 (2001); Sharp, Genes Dev 15: 485-490 (2001), Waterhouse et al., Proc. Natl. Acad. Sci. USA 95(23): 13959-13964 (1998) all of which are incorporated herein by reference in their entireties and at least form material related to delivery and making of RNAi molecules. The RNAi agents disclosed in U.S. Published Application No. 2003/0228601 and International Publication No. WO2004/0798950, which are incorporated by reference with respect to the different RNAi agents, can also be used herein.

e. Attachment of Biomolecule to Support

Once the first polymer has been attached to the substrate, one or more biomolecules can be attached to the first polymer using the techniques presented above. In a further aspect, when the biomolecule is a nucleic acid, the nucleic acid can be printed on the first polymer using techniques known in the art. The amount of biomolecule that can be attached to the polymer layer can vary depending upon among other things, for example, the biomolecule and first polymer selected and the analyte to be detected. In a further aspect, one or more different biomolecules can be placed at different locations on the support. In the case when different biomolecules are used, the biomolecules can be printed at the same time or different time.

In a further aspect, the spotting solution (i.e., ink) containing the biomolecule also contains optional components such as, for example, an alkylene diol, a betaine, a detergent, a salt, or an aprotic solvent. The selection of components present in the ink formulation will vary and can be used to control spot size.

The term “alkylene diol” as used herein is any compound that possesses two hydroxyl groups and at least one CH₂ group. The alkylene diol can be branched or straight chain. In one aspect, the alkylene diol comprises the formula HO(CH₂)ₙOH, wherein n is an integer of from 1, 2, 3, 4, 5, 6, 7, 8, 9, 10, 11, 12, 13, 14, 15, 16, 17, 18, 19, 20, 21, 22, 23, 24, or 25. In a further aspect, the alkylene diol comprises a straight chain compound such as, for example, methylene glycol, ethylene glycol, propylene glycol, butylene glycol, or a mixture thereof. In a further aspect, the alkylene diol comprises a branched compound such as, for example, isopropyl diol, isobutyl- and sec-butyl diol, neopentyl diol, and the like. In a further aspect, the alkylene diol is from 30 to 70% by volume of the composition. In another aspect, the alkylene diol is 30%, 35%, 40%, 45%, 50%, 55%, 60%, 65%, or 70% by volume of the composition, wherein any value can form a lower and upper endpoint. In one aspect, the alkylene diol is from 40 to 60% by volume of the composition.

Detergents useful herein include, but are not limited to, a surfactant. A “surfactant” as used herein is a molecule composed of hydrophilic and hydrophobic groups (i.e., an amphiphile). Suitable surfactants can be generally classified as ionic (anionic/cationic/dipolar) and nonionic. In a further aspect, polymeric surfactants, natural surfactants, silicon surfactants, fluorinated surfactants, oligomeric surfactants, dimeric surfactants, and the like, are suitable for the compositions and methods disclosed herein. In a further aspect, the surfactants disclosed in U.S. Pat. No. 6,849,426, which is incorporated by reference in its entirety, can be used herein.

In a further aspect, the detergent comprises an anionic surfactant. Any anionic surfactants known in the art can be used herein. In a further aspect, the anionic surfactant comprises an alkyl aryl sulfonate, an alkyl sulfate, or sulfated oxyethylated alkyl phenol. In a further aspect, the anionic surfactant can be an alkylbenzene sulfonate (detergent), a fatty acid based surfactant, a lauryl sulfate (e.g., a foaming agent), a di-alkyl sulfosuccinate (e.g., a wetting agent), a lignosulfonate (e.g., a dispersant), and the like, including mixtures thereof. In other examples, linear alkylbenzene sulphonic acid, sodium lauryl ether sulphate, alpha olefin sulphonates, phosphate esters, sodium sulphosuccinates, hydrotropes, and the like, including mixtures thereof, can be used. In a further aspect, the anionic surfactant comprises sodium dodecylbenzene sulfonate, sodium decylbenzene sulfonate, ammonium methyl dodecylbenzene sulfonate, ammonium dodecylbenzene sulfonate, sodium octadecylbenzene sulfonate, sodium nonylbenzene sulfonate, sodium dodecylnaphthalene sulfonate, sodium hetadecylbenzene sulfonate, potassium eicososyl naphthalene sulfonate, ethylamine undecylnaphthalene sulfonate, sodium docosylnaphthalene sulfonate, sodium octadecyl sulfate, sodium hexadecyl sulfate, sodium dodecyl sulfate, sodium nonyl sulfate, ammonium decyl sulfate, potassium tetradecyl sulfate, diethanolamino octyl sulfate, triethanolamine octadecyl sulfate, ammonium nonyl sulfate, ammonium nonylphenoxyl tetraethylenoxy sulfate, sodium dodecylphenoxy triethyleneoxy sulfate, ethanolamine decylphenoxy tetraethyleneoxy sulfate, or potassium octylphenoxy triethyleneoxy sulfate.

In a further aspect, the detergent comprises a nonionic surfactant. Any nonionic surfactant can be used. Suitable nonionic surfactants do not ionize in aqueous solution, because their hydrophilic group is of a non-dissociable type, such as alcohol, phenol, ether, ester, or amide. They can be classified as ethers (e.g., polyhydric alcohols such as glycerin, sorbitole, sucrose, etc.), fatty acid esters (e.g., glycerin fatty acid ester, sorbitan fatty acid ester, sucrose fatty acid ester, etc.), esters (e.g., compounds made by applying, for example, ethylene oxide to a material having hydroxyl radicals such as high alcohol, alkyl-phenol, and the like), ether/esters (e.g., compounds made by applying, for example, the ethylene oxide to the fatty acid or polyhydric alcohol fatty acid ester, having both ester bond and ether bond in the molecule), and other types (e.g., the fatty acid alkanol-amide type or the alkylpolyglyceride type). Other suitable examples of nonionic surfactants can include, but are not limited to, alcohol ethoxylates and alkyl phenol ethyoxylates, fatty amine oxides, alkanolamides, ethylene oxide/propylene oxide block copolymers, alkyl amine ethoxylates, tigercol lubricants, etc. In a further aspect, the nonionic surfactant comprises the condensation product between ethylene oxide or propylene oxide with the propylene glycol, ethylene diamine, diethylene glycol, dodecyl phenol, nonyl phenol, tetradecyl alcohol, N-octadecyl diethanolamide, N-dodecyl monoethanolamide, polyoxyethylene sorbitan monooleate, or polyoxyethylene sorbitan monolaurate.

In a further aspect, the detergent comprises a cationic surfactant. Any cationic surfactant known in the art can be used herein. Suitable cationic surfactants included, but are not limited to, quaternary ammonium compounds, imidazolines, etc. Such cationic surfactants can be obtained commercially or can be prepared by methods known in the art. In a further aspect, the cationic surfactant comprises ethyl-dimethylstearyl ammonium chloride, benzyl-dimethyl-stearyl ammonium chloride, benzyldimethyl-stearyl ammonium chloride, trimethyl stearyl ammonium chloride, trimethylcetyl ammonium bromide, dimethylethyl dilaurylammonium chloride, dimethyl-propyl-myristyl ammonium chloride, or the corresponding methosulfate or acetate.

Other examples of suitable surfactant include natural surfactants, which can have their source from plant or animal organs. In another example, a bolaform surfactant can be used. A bolaform surfactant is a surfactant that has two hydrophilic head groups at opposite ends of a hydrophobic tail. In a further aspect, the detergent can be Tween-20 or Triton x-100.

In a further aspect, the detergent comprises an organic acid or the salt thereof. Examples of organic acids useful herein include saturated or unsaturated fatty acids. In one aspect, the organic acid comprises the formula CH₃(CH₂)ₘCO₂H, wherein m is 1, 2, 3, 4, 5, 6, 7, 8, 9, 10, 11, 12, 13, 14, 15, 16, 17, 18, 19, 20, 21, 22, 23, 24, or 25, or the salt thereof. In a further aspect, the detergent comprises hexanoic acid, heptenoic acid, octanoic acid, nonanoic acid, decanoic acid, or the salt thereof. In one aspect, when the detergent comprises an organic acid such as, for example, octanoic acid, the amount of organic acid can be 0.005%, 0.01%, 0.02%, 0.03%, 0.04%, 0.05%, 0.06%, 0.07%, 0.08%, 0.09%, 0.10%, 0.15%, 0.20%, or 0.25% by volume of the composition, where nay value can form a lower and upper endpoint of the concentration range. In a further aspect, the amount of organic acid is from 0.05% to 0.10% by volume of the composition.

It is contemplated that mixtures of surfactants can also be used herein.

It is contemplated that the spotting solution or ink containing the biomolecule can include a salt. In a further aspect, the salt can be an organic salt, an inorganic salt, or a mixture thereof. In a further aspect, the organic salt comprises a citrate. In a further aspect, the inorganic salt comprises NaCl, KCl, MgCl₂, LiCl, or a mixture thereof. In another aspect, the salt comprises a mixture of NaCl and sodium citrate.

The pH of the ink can also vary depending upon among other things the selection of starting materials and the biomolecule to be spotted, which can be readily determined by one of ordinary skill in the art. In a further aspect, the spotting composition comprises an alkaline pH. In another aspect, the pH is greater than 7.0. In a further aspect, the pH is 7.0, 8.0, 8.5, 9.0. 9.5, 10.0, 10.5, 11.0, 11.5, 12.0, 12.5, or 13, where any pH value can form a lower and upper end-point for a pH range. The pH of the ink can be adjusted by adding bases such as, for example, hydroxides, carbonates, phosphates, and the like.

The ink can also contains solvents as well that can reduce the rate of evaporation of water once the biomolecule has been spotted on the support. Examples of solvents useful herein include, but are not limited to, dimethylsulfoxide, polyethylene glycol, ethylene glycol, glycerol, or dextran.

In a further aspect, prior to attaching the biomolecule to the first polymer, the biomolecule is in a solution comprising an aprotic solvent (e.g., DMSO), a surfactant (e.g., SDS), and a buffer, wherein the pH of the solution is from 7 to 10, 7 to 9, 7 to 8, 8 to 10, or 9 to 10.

In a further aspect, the biomolecule can be deposited on (i.e., attached to) the support by immersing the tip of a pin into the composition comprising the biomolecule; removing the tip from the composition, wherein the tip comprises the composition; and transferring the composition to the support. This aspect can be accomplished, for example, by using a typographic pin array. The depositing step can be carried out using an automated, robotic printer. Such robotic systems are available commercially from, for example, Intelligent Automation Systems (IAS), Cambridge, Mass.

The pin can be solid or hollow. The tips of solid pins are generally flat, and the diameter of the pins determines the volume of fluid that is transferred to the substrate. Solid pins having concave bottoms can also be used. In one aspect, to permit the printing of multiple arrays with a single sample loading, hollow pins that hold larger sample volumes than solid pins and therefore allow more than one array to be printed from a single loading can be used. Hollow pins include printing capillaries, tweezers and split pins. An example of a preferred split pen is a micro-spotting pin that TeleChem International (Sunnyvale, Calif.) has developed. In one aspect, pins made by Point Tech can be used herein. The spotting solutions described herein can be used in a number of commercial spotters including, but not limited to, Genetix and Biorobotics spotters.

In a further aspect, once the biomolecule has been attached (i.e., spotted) on the support, the support can be exposed to a relative humidity of greater than 40%. Not wishing to be bound by theory, it is believed that when the support with the attached biomolecule is exposed to humidity, spot shadows are reduced. By reducing spot shadows, better sensitivity and detection of an analyte is possible. In a further aspect, the biomolecule is attached to the first polymer under a relative humidity of from about 40%, 45%, 50%, 55%, 60%, 65%, 70%, 75%, 80%, 85% to about 90%, where any value can form an endpoint of a range. In a further aspect, the biomolecule is attached to the first polymer under a relative humidity of from about 55% to 80% or from about 60% to about 75%. The duration of humidity exposure can vary from 0.5, 1, 2, 3, 4, 5, 6, 7, 8, 9, 10, 11, 12, 13, 14, 15, 16, 17, 18, 19, 20, 21, 22, 23, or 24 hours, where any value can form an endpoint of a range. In a further aspect, the biomolecule is attached to the first polymer under a relative humidity of from 50% to 85% from 2 hours to 24 hours, or from 65% to 80% of from 8 hours to 16 hours.

It is also contemplated that during attachment of the biomolecule to the support, the attachment can be performed under humid conditions. This can help control spot size, reduce evaporation of water present in the ink, and reduce printing runs.

After exposure of the support to a relative humidity greater than 40%, a blocking agent is attached to the first polymer. Inadequate blocking can lead to high levels of non-specific binding to the surface, making analysis of results difficult if not impossible. Thus, in one aspect, all reactive sites present on the first polymer that are not attached to the biomolecule are reacted with a blocking agent to render the active site inactive to the analyte to be detected. Here, the analyte will interact with the biomolecule and not the first polymer, which leads to increased specific binding and better detection of the analyte.

In a further aspect, the blocking agent comprises at least one nucleophilic group, the first polymer comprises at least one electrophilic group, and the blocking agent is attached to the first polymer by a reaction between the electrophilic group and the nucleophilic group. In a further aspect, the blocking agent is covalently attached to the first polymer. For example, when the blocking agent comprises an amine group, it can react with an electrophilic group present on the first polymer (e.g., an epoxy, anhydride, ester group) to produce a covalent bond. In another aspect, when the blocking agent possesses a group that can be converted to a charged group (i.e., a salt), then the blocking agent can form an electrostatic bond with the first polymer.

In a further aspect, the blocking agent comprises 2-(2-aminoethoxy)ethanol, N,N-dimethyl ethylenediamine, ethanolamine, ethylenediamine, 4,7,10-trioxa-1,13-tridecanediamine, PEG amine, Tris hydrochloride, diethylaminoethyl-cellulose, diethylaminoethyl-cellulose amine, diethylaminoethyl-cellulose dextran, bovine serum albumin, chicken egg albumin, dry milk, pluronic or any combination thereof. In a further aspect, when the blocking agent is PEG amine, the PEG amine has a molecular weight of from 400 to 100,000 Da. In a further aspect, the blocking agent comprises 2-(2-aminoethoxy)ethanol or N,N-dimethyl ethylenediamine in a solution of pH from 7 to 10, 7 to 9, 7 to 8, 8 to 10, or 9 to 10. For example, the blocking agent can be in a solution of base such as, for example, sodium hydroxide or sodium borate.

In a further aspect, the substrate is glass, the tie layer derived from gamma-aminopropylsilane, the first polymer is poly(styrene-co-maleic anhydride), the biomolecule is an oligonucleotide, wherein the relative humidity is from about 60% to about 75%, and the blocking agent is 2-(2-aminoethoxy)ethanol or N,N-dimethyl ethylenediamine.

II. Methods of Use

Described herein are methods for assaying a sample comprising one or more analytes. In one aspect, described herein is a method for performing an assay of a sample comprising an analyte, the method comprising:

a. attaching a biomolecule to an article, wherein the article comprises a substrate, a first tie layer attached to the substrate, a first polymer attached to the first tie layer, wherein the biomolecule attaches to the first polymer;

b. exposing the article with the attached biomolecule to a relative humidity of greater than 40%;

c. attaching a blocking agent to the first polymer;

d. contacting support with the sample comprising the analyte, wherein after the contacting step, the analyte is bound to the biomolecule; and

e. detecting the bound analyte.

In another aspect, described herein is a method for method for performing an assay of a sample comprising an analyte, the method comprising:

a. attaching a biomolecule to an article, wherein the article comprises a substrate comprising a first polymer, wherein the biomolecule attaches to the first polymer;

b. exposing the article with the attached biomolecule to a relative humidity of greater than 40%;

c. attaching a blocking agent to the first polymer;

d. contacting support with the sample comprising the analyte, wherein after the contacting step, the analyte is bound to the biomolecule; and

e. detecting the bound analyte.

Any of the supports described herein with one or more biomolecules attached thereto can be used to assay an analyte upon contact of the analyte with the support. Upon contact of the analyte with the support, a chemical interaction between the biomolecule and the analyte occurs to produce a bound analyte; however, it is possible that an interaction may occur to some extent between the first polymer and the analyte. The nature of the interaction between the biomolecule and the analyte will vary depending upon the biomolecule and the analyte selected. In one aspect, the interaction between the biomolecule and the analyte can result in the formation of an electrostatic bond, a hydrogen bond, a hydrophobic bond, or a covalent bond. In another aspect, an electrostatic interaction can occur between the biomolecule and the analyte.

The analyte can be any naturally-occurring or synthetic compound. Examples of analytes that can be bound to the biomolecules on the substrate include, but are not limited to, a drug, an oligonucleotide, a nucleic acid, a protein, a peptide, an antibody, an antigen, a hapten, or a small molecule (e.g., a pharmaceutical drug). Any of the biomolecules described above can be an analyte for the methods described herein. In one aspect, a solution of one or more analytes is prepared and added to one or more wells that have a biomolecule attached to the outer surface of the microplate. In this aspect, it is contemplated that different biomolecules can be attached to different wells of the microplate; thus, it is possible to detect a number of different interactions between the different biomolecules and the analyte. In one aspect, a protein can be immobilized on the microplate to investigate the interaction between the protein and a second protein or small molecule. Alternatively, a small molecule can be immobilized on the microplate using the techniques described herein to investigate the interaction between the small molecule and a second small molecule or protein. In a further aspect, the biomolecule can be an oligonucleotide that can hybridize a second oligonucleotide (i.e., analyte). In a further aspect, when the substrate is a microplate, the assay can be a high-throughput assay.

In a further aspect, an enhancer can be used to facilitate specific binding between the analyte and the biomolecule. In one aspect, the enhancer can be added to a solution of the analyte prior to contact with the support. In a further aspect, the enhancer is a low molecular weight, water soluble polymer. In a further aspect, the enhancer comprises a low molecular weight (e.g., 5,000 to 200,000 Da, 5,000 to 150,000 Da, 5,000 to 100,000, Da, or 8,000 to 100,000 Da) poly(vinylpolypyrrolidone) polymer, low molecular weight (e.g., 3,000 to 200,000 Da, 3,000 to 150,000 Da, 3,000 to 100,000 Da, or 5,000 to 50,000 Da) dextran sulfate, Ficoll (e.g., 5,000 to 800,000 Da, 10,000 to 500,000 Da, 20,000 to 400,000 Da, 30,000 to 300,000 Da, 50,000 to 200,000 Da, or 80,000 to 100,000 Da), low molecular weight (e.g., 3,000 to 200,000 Da, 3,000 to 100,000 Da, 3,000 to 50,000, or 5,000 to 50,000 Da) polyethylene glycol, or any combination thereof. In a further aspect, the enhancer comprises poly(vinylpolypyrrolidone) having a molecular weight of about 10 kDa (PVP10), about 29 kDa (PVP29), about 50 kDa (PVP50), or about 55 kDa (PVP55), polyethyleneglycol having a molecular weight of about 8 kDa, or dextran sulfate having a molecular weight of about 8 kDa.

The amount of enhancer used can vary depending upon the selection of the enhancer, the analyte, and the support used. In a further aspect, the enhancer is present in the sample at from about 0.1%, 0.5%, 1%, 1%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% by volume. In a further aspect, the enhancer is present in the sample at from about 0.1% to about 30% by volume, about 1% to about 25%, about 1% to about 20%, about 1% to about 15%, about 2% to about 15%, about 3% to about 15%, about 3% to about 10%, about 5% to about 10%, or about 3% to about 4% by volume of the sample. It is contemplated that other components can be present in the sample of the analyte including, but not limited to, salts and buffers. In one aspect, the sample contains a salt at a concentration of from 0.5×SSC to 20×SSC, 0.5×SSC to 15×SSC, 1×SSC to 10×SSC, 2×SSC to 9×SSC, or 2×SSC to 8×SSC.

In a further aspect, an array can be used in any of the methods described herein. In one aspect, the array comprises a plurality of biomolecules on the substrate, wherein the biomolecules are on discrete and defined locations on the support. Arrays have been used for a wide range of applications such as gene discovery, disease diagnosis, drug discovery (pharmacogenomics) and toxicological research (toxicogenomics). An array is an orderly arrangement of biomolecules. The typical method involves contacting an array of biomolecules with a target of interest to identify those compounds in the array that bind to the target. Arrays are generally described as macro-arrays or micro-arrays, the difference being the size of the sample spots. Macro-arrays contain sample spot sizes of about 300 microns or larger whereas micro-arrays are typically less than 200 microns in diameter and typically contain thousands of spots. In one aspect, the distance between each biomolecule in the array can be from 200 to 500 μm.

Methods for producing arrays are known in the art. For example, Fodor et al., 1991, Science 251:767-773 describe an in situ method that utilizes photo-protected amino acids and photo lithographic masking strategies to synthesize miniaturized, spatially-addressable arrays of peptides. This in situ method has recently been expanded to the synthesis of miniaturized arrays of oligonucleotides (U.S. Pat. No. 5,744,305). Another in situ synthesis method for making spatially-addressable arrays of immobilized oligonucleotides is described by Southern, 1992, Genomics 13:1008-1017; see also Southern & Maskos, 1993, Nucl. Acids Res. 21:4663-4669; Southern & Maskos, 1992, Nucl. Acids Res. 20:1679-1684; Southern & Maskos, 1992, Nucl. Acids Res. 20:1675-1678. In this method, conventional oligonucleotide synthesis reagents are dispensed onto physically masked glass slides to create the array of immobilized oligonucleotides. U.S. Pat. No. 5,807,522 describes a deposition method for making micro arrays of biological samples that involves dispensing a known volume of reagent at each address of the array by tapping a capillary dispenser on the substrate under conditions effective to draw a defined volume of liquid onto the substrate.

In one aspect, an array of nucleic acid(s) can be printed on any of the substrates described herein. The techniques disclosed in U.S. Published Application No. 2003/0228601 to Sabatini can be used herein, which is incorporated by reference with respect to the different arrays and nucleic acid libraries that can be used in the methods described herein.

Once the support with the attached biomolecules(s) has been contacted with the analyte, the bound analyte is detected. As described above, one of the advantages of the substrates described herein is that non-specific binding of the analyte is reduced.

In a further aspect, the bound analyte is labeled for detection purposes. Depending upon the detection technique used, in one aspect, the analyte can be labeled with a detectable tracer prior to detection. The interaction between the analyte and the detectable tracer can include any chemical or physical interaction including, but not limited to, a covalent bond, an ionic interaction, or a Lewis acid-Lewis base interaction. A “detectable tracer” as referred to herein is defined as any compound that (1) has at least one group that can interact with the analyte as described above and (2) has at least one group that is capable of detection using techniques known in the art. In a further aspect, the analyte can be labeled prior to contacting the support. In another aspect, the analyte can be labeled after it has been contacted with the support. Examples of detectable tracers include, but are not limited to, fluorescent and enzymatic tracers.

In another aspect, detection of the bound analyte can be accomplished with other techniques including, but not limited to, fluorescence, phosphorescence, chemilumenescence, bioluminescence, Raman spectroscopy, optical scatter analysis, mass spectrometry, etc. and other techniques generally known to those skilled in the art. In a further aspect, the bound analyte is detected by label-independent detection or LID. Examples of LID include, but are not limited to, surface plasmon resonance or a resonant waveguide gratings (e.g. Corning LID system).

In summary, the methods and techniques described herein efficiently reduce non-specific binding of biomolecules to the substrate surfaces and significantly improve signal to background ratio and overall assay quality. The methods also increase array signal intensity, sensitivity and assay quality in a timely and economical manner and further improve the assay specificity.

### EXAMPLES

The following examples are put forth so as to provide those of ordinary skill in the art with a complete disclosure and description of how the materials, articles, and methods described and claimed herein are made and evaluated, and are intended to be purely exemplary and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

Material and Methods

SMA slides. Corning Incorporated GAPS slides were coated with a solution of poly(styrene-co-maleic anhydride) (SMA) in 1-methoxy-2-propanol acetate. The coating can be carried out either through spin coating or dip coating. The thickness of the SMA coating was mainly controlled by the SMA concentration. The coated slides had gone through quality control with respect to the autofluorescence (<200 at 750 PMT) and uniformity of the surface.

Test Arrays. A total of 48 oligonucleotide samples were used to create the test arrays for internal and alpha test sites. These samples included 42 human genes as positive controls, 3 arabidopsis genes and 3 inks only as negative controls. All positive and negative oligonucleotides were custom-synthesized by Qiagen [Valencia, Calif.] with column-desalt purification. All oligos were C6-terminal amine-modified except for 5 sets of positive genes that were synthesized with no amine modification. The samples were prepared in an ink containing 40% DMSO/80 mM sodium borate buffer (pH9.2)/0.01% SDS at 500 ng/μl and printed in a 12×4 layout and repeated four times per slide unless stated otherwise with a Cartesian printer using Telechem ChipMaker microspotting pins.

Arrayed slide processing. After the slides were printed with the alpha test array, they were kept in an enclosed clean environment at 70% RH for a maximum of 17 hours unless stated otherwise. The arrayed slides were blocked in 100 ml of 100 mM AEE [2-(2-aminoethoxy)ethanol] in 150 mM sodium borate pH9.2/0.1% SDS at 42° C. for 10 minutes and then twice in 1×SSC at room temperature for 1 minute unless otherwise stated. The arrayed slides were then prehybridized with 2×SSC/0.05% SDS/0.2% BSA at 42° C. for 15 minutes, and then twice in 0.2×SSC at room temperature for 1 minute. The slides were dried by centrifugation at 2000 rpm for 1 minute.

Total RNA or mRNA was labeled using common techniques of microarray technologies. Cy3 or Cy5 labeled cDNA was added to the hybridization buffer in a final volume of 60 μl. The arrays were hybridized at 42° C. over night.

The array slides were washed with 2×SSC/0.05% SDS at 42° C. for 15 minutes; 1×SSC at room temperature for 5 min twice; then 0.2×SSC at room temperature for 1 minute twice. The slides were dried at 2000 rpm for 1 minute. Slides were scanned at an appropriate PMT setting.

Results and Discussion

Improvement of Microarray Hybridization Quality with High Efficiency Blocking Reagents

An extensive effort was made to develop efficient blocking methods on the SMA array surface. A number of small compounds with different numbers of amine groups, different lengths and charges were investigated. Ethanolamine (EA) and 2-(2-aminoethoxy)ethanol (AEE) are not charged and contain an amine group that can form covalent bonds with the SMA anhydride groups and were tested as blocking reagents for the SMA surface. Different numbers of amine groups per molecule e.g. PEG1900 amine, ethylene diamine (EDA), N,N-dimethyl ethylenediamine (DMEDA), and 4,7,10-trioxa-1,13-tridecanediamine (TODA) were evaluated for blocking efficiency. Theoretically the more number of amine group in a single molecule, the higher potential it has of forming multiple and strong covalent attachment points. Polyethylene glycol (PEG) and diethylaminoethyl-cellulose (DEAE) coated surfaces are usually hydrophilic. By applying either of them to the SMA surface it should increase the surface hydrophilicity. Large molecules such as DEAE amine, DEAE dextran, bovine serum albumin (BSA), and surfactant pluronic L122 (BASF Corporation, NJ, USA) potentially occupy a larger surface area while attaching onto the surface.

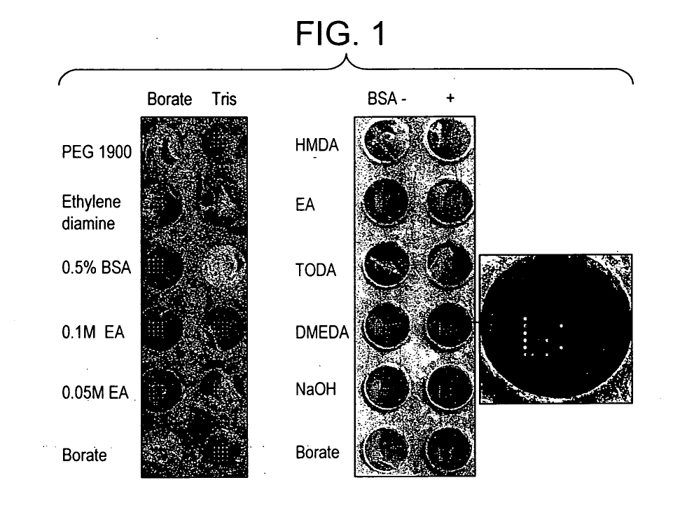

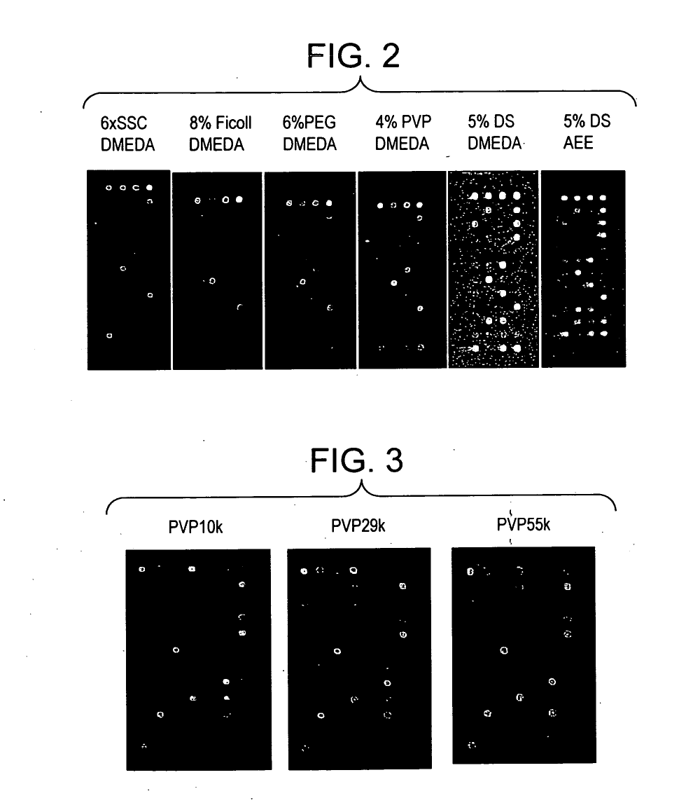

Blocking reagents were separately prepared in either 100 mM Tris-HCl (pH 9) or 100 mM sodium borate (pH 9). The effect of the different blockers on microarray hybridization was evaluated by way of hybridization data. Hybridization data indicated the different blockers showed a quite different quality of hybridization image ranging from very bad to good images (FIG. 1). Among them, AEE and DMEDA yield the least non-specific binding. Non-specific binding, however, was still quite high with the hybridization buffer of 5% dextran sulfate/1×SSC/0.1% SDS/0.2% BSA even with AEE as blocker (FIG. 2).

The Effect of Organic Polymers on Array Hybridization

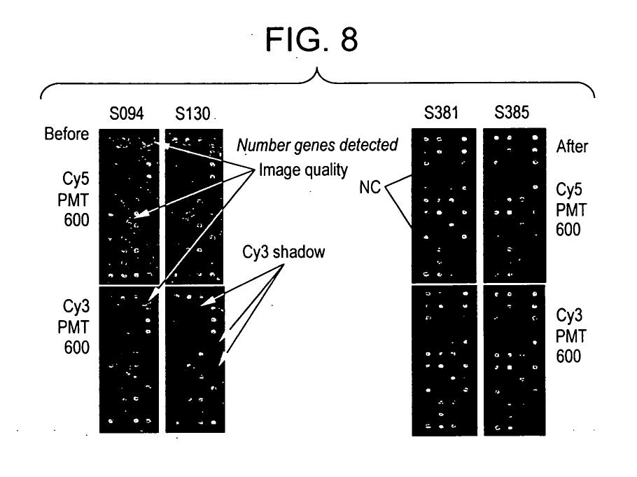

High molecular weight dextran sulfate (DS) is commonly used in the Southern or Northern hybridization protocol to increase the hybridization signal (Chang et al, 1974, Wahl et al, 1979). However, only a few DNA microarray assay methods have suggested the use of DS during hybridization. The addition of DS resulted in DNA aggregation and precipitation at low temperature and high DNA concentrations, leading to an uneven and high fluorescent background (see also FIG. 8A). Also, the addition of DS reduced the stringency of hybridization, and as a result, decreased the specificity of hybridization. The same blocking and hybridization processes using no DS showed clean hybridization images although the signal intensity was very low. This result suggested it is possible to achieve a clean hybridization image by abandoning the DS in the hybridization buffer.

Several organic polymers were evaluated as potential concentration enhancers in the array hybridization buffer. PVP360, Ficoll400, and polyethylene glycol [MW8 kDa] (PEG8) were investigated under different concentrations for a total of 28 different combinations (Table 1). DMEDA and AEE were used as blockers (FIG. 2). Preliminary hybridization data indicated the useful concentration ranges of DS500, Ficoll400, PEG8 and PVP360 for array hybridization were, respectively 3-5%, 5-10%, 6-10% and 3-4%. The hybridization buffer with Ficoll400, PEG8 and PVP360 showed a much cleaner hybridization image albeit lower signal than with DS (FIG. 2). At 4% of PVP360, a reasonable hybridization signal and low background were achieved.

TABLE 1

| | | | | | | polyA |
|---|---|---|---|---|---|---|
| Hybe# | DS500 | Ficoll400 | PEG8 | PVP360 | SSC | (ng/μl) |
| 1. | 0 | 0.5% | 0 | 0 | 1× | 100 |
| 2. | 0 | 1% | 0 | 0 | 1× | 100 |
| 3. | 0 | 3% | 0 | 0 | 1× | 100 |
| 4. | 0 | 5% | 0 | 0 | 1× | 100 |
| 5. | 0 | 6% | 0 | 0 | 1× | 100 |
| 6. | 0 | 8% | 0 | 0 | 1× | 100 |
| 7. | 0 | 0 | 0.5% | 0 | 1× | 100 |
| 8. | 0 | 0 | 1% | 0 | 1× | 100 |
| 9. | 0 | 0 | 3% | 0 | 1× | 100 |
| 10. | 0 | 0 | 5% | 0 | 1× | 100 |
| 11. | 0 | 0 | 6% | 0 | 1× | 100 |
| 12. | 0 | 0 | 8% | 0 | 1× | 100 |
| 13. | 0 | 0 | 0 | 0.2% | 1× | 100 |
| 14. | 0 | 0 | 0 | 0.5% | 1× | 100 |
| 15. | 0 | 0 | 0 | 1% | 1× | 100 |
| 16. | 0 | 0 | 0 | 2% | 1× | 100 |
| 17. | 0 | 0 | 0 | 3% | 1× | 100 |
| 18. | 0 | 0 | 0 | 3.25% | 1× | 100 |
| 19. | 0 | 0 | 0 | 0 | 6× | 100 |
| 20. | 5% | 0 | 0 | 0 | 1× | 100 |
| 21. | 5% | 0 | 0 | 0 | 1× | 0 |
| 22. | 4.5% | 0 | 0 | 0 | 1× | 0 |
| 23. | 4% | 0 | 0 | 0 | 1× | 0 |
| 24. | 3.5% | 0 | 0 | 0 | 1× | 0 |
| 25. | 3% | 0 | 0 | 0 | 1× | 0 |
| 26. | 3% | 0 | 0 | 0 | 2× | 0 |
| 27. | 3% | 0 | 0 | 0 | 3× | 0 |
| 28. | 3% | 0 | 0 | 0 | 4× | 0 |

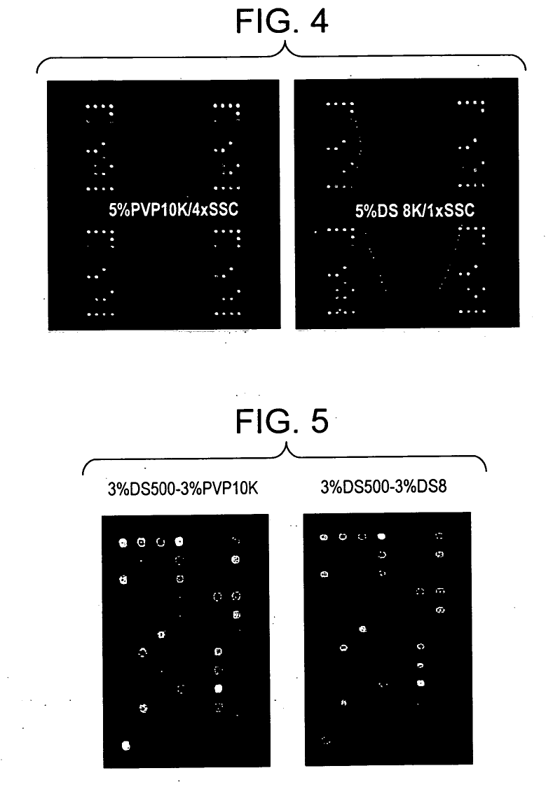

Next, a low molecular weight PVP polymer [MW 10 kDa] (PVP10) was investigated using AEE as the blocker and 6×SSC and 0.1% SDS as the hybridization buffer. At 5% and 10% PVP 10, clean hybridization images were achieved. 5% PVP performed as good as 10% PVP. PVP10 [MW 10 kDa], PVP29 [MW 29 kDa], and PVP55 [MW 55 kDa] showed very comparable hybridization images (FIG. 3). The data indicated that 5% PVP10/6×SSC/0.1% SDS showed high signal intensity and clean hybridization images. Since high molecular weight dextran sulfate is commonly used in Southern hybridization but does not work in microarray hybridization on SMA, the use of low molecular weight DS was investigated. Low molecular weight DS8 [MW 8 kDa of dextran sulfate] also showed clean hybridization images. However, PVP10 showed the best result in the studies (FIG. 4).

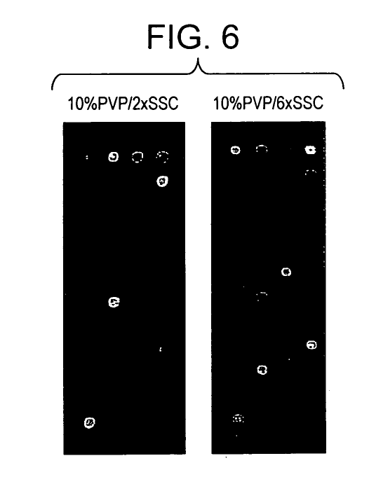

The possibility of using a combination of 3% PVP 10 plus 3% DS500 or 3% DS8 plus 3% DS500 was further evaluated since DS500 tends to show high hybridization signals. The former combination showed high quality hybridization data whereas the latter had high background (FIG. 5). After balancing the hybridization quality, signal intensity and cost, PVP10 or 29 was used instead of the PVP/DS combination. By varying the SSC salt concentrations, it was discovered that 5% PVP29/6×SSC showed better result than 5% PVP29/2×SSC without sacrificing the assay specificity (FIG. 6).

Humidity and Spotting Ink on Array Performance

During early stage assay development, there were also spot shadow issues with the sodium borate ink on the SMA surface, especially at the Cy3 channel (FIG. 8A). After tracking each stage of the assay process, it was realized that the spot shadow appeared after the arrayed slides were treated at 85% relative humidity (RH) for overnight (˜17 to 20 hours). It was predicted that high humidity, which increases the chemical reaction rate and enhances oligo covalent attachment, causes the spotted oligos to move around. Thus, high humidity may cause the spot shadow. To test this, the SMA slides were spotted with 6 different inks and scanned after spotting but before humidity treatment. There was no shadow spot on the slides. The slides were then separately put into 45%, 70% and 85% RH chambers for overnight and scanned again. There were severe spot shadows from the slides at 70% and 85% RH, especially with the ink consisting of 20% DMSO/150 mM sodium borate/0.01% SDS. It indicated that the combination of high humidity and spotting ink contributed to the shadow spots on SMA surface. It was reasoned that high humidity improves the covalent immobilization and, thus, the array performance.

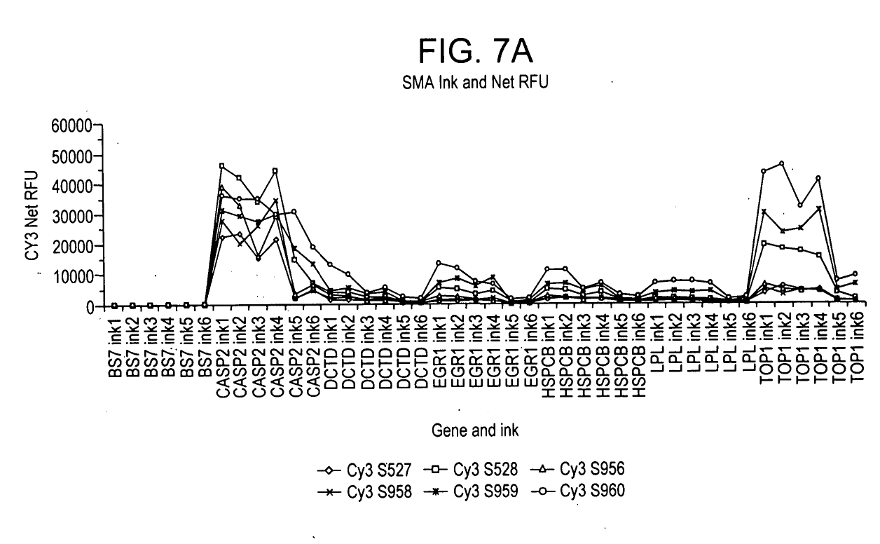

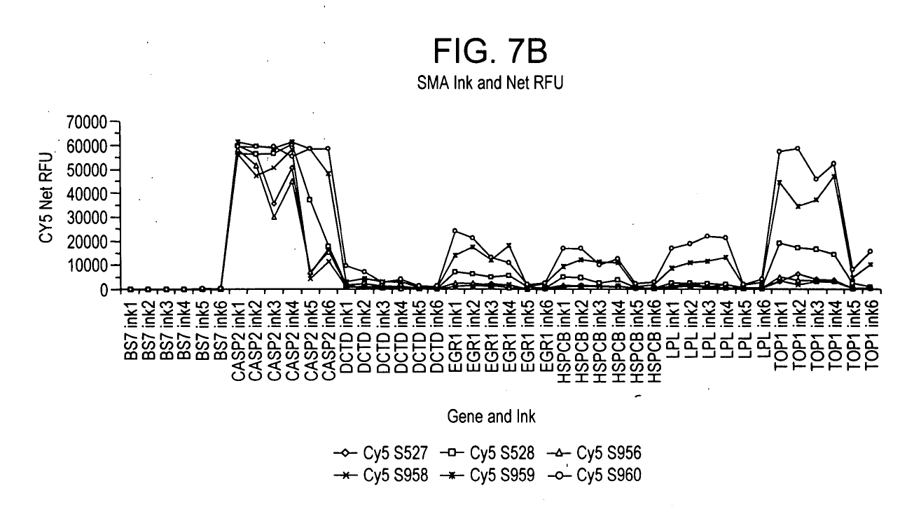

The effects of the post printing humidity and spotting ink on the hybridization results were further investigated. Hybridization data suggested that the high humidity contributed to the high hybridization signal (FIG. 7). The array performance under 85% RH was also compared with that under 70% RH with the spotting ink composed of 40% DMSO/80 mM sodium borate buffer (pH9.2)/0.01% SDS. The hybridization data indicated that those arrays performed similarly at either 70% or 85% relative humidity treatment. The combination of high humidity and the ink composition of 20% DMSO/120 mM sodium borate/0.01% SDS caused the shadow spots on the SMA surface. After screening the inks, the ink composed of 40% DMSO/80 mM sodium borate/0.01% SDS produced a consistently high hybridization signal and no obvious spot shadows. The spot shadows disappear with the right ink components and humidity.

Throughout this application, various publications are referenced. The disclosures of these publications in their entireties are hereby incorporated by reference into this application in order to more fully describe the compounds, compositions and methods described herein.

Various modifications and variations can be made to the materials, methods, and articles described herein. Other aspects of the materials, methods, and articles described herein will be apparent from consideration of the specification and practice of the materials, methods, and articles disclosed herein. It is intended that the specification and examples be considered as exemplary.

### REFERENCES

“Effects of microscopic and macroscopic viscosity on the rate of renaturation of DNA,” Chang C T, Hain T C, Hutton J R, and Wetmur J G., Biopolymers, 1974, 13, 1847-58.
“Exploring the metabolic and genetic control of gene expression on a genomic scale,” DeRisi J L, Iyer V R, Brown P O, Science, 1997, 278, 680-686.
“Genome-wide screening for trait conferring genes using DNA microarrays,” Gill R T, Wildt S, Yang Y T, Ziesman S, Stephanopoulos G., Proc Natl Acad Sci USA., 2002, 99, 7033-7038.
“A concise guide to cDNA microarray analysis,” Hegde, P.; Qi, R.; Abernathy, K.; Gay, C.; Dharap, S.; Gaspard, R.; Earle-Hughes, J.; Snesrud, E.; Lee, N.; Quackenbush, J., Biotechniques 2000, 29, 548-556.
“Analysis of repeatability in spotted cDNA microarrays,” Jenssen T K, Langaas M, Kuo W P, Smith-Sørensen B, Myklebost O, Hovig E., Nucleic Acids Res., 2002, 30, 3235-3244.
“Normalization and analysis of DNA microarray data by self-consistency and local regression,” Kepler T B, Crosby L, Morgan K T., Genome Biol. 2002, 3, 1-12.
“Comprehensive copy number and gene expression profiling of the 17q23 amplicon in human breast cancer,” Monni O, Bärlund M, Mousses S, Kononen J, Sauter G, Heiskanen M, Paavola P, Avela K, Chen Y, Bittner M L, Kallioniemi A. Proc Natl Acad Sci USA., 2001, 98, 5711-5716.
“High resolution analysis of DNA copy number changes in the human genome with oligonucleotide microarrays,” Li G, Bao P, Lai F, Xie Y, Muller U R., Appl. Genomics Proteomics, 2003, 2, 93-100.
“How many replicates of arrays are required to detect gene expression changes in microarray experiments? A mixture model approach,” Pan W, Lin J, Le C T, Genome Biol. 2002, 3, 1-10.
“Immobilization of Protein Ligands with Methyl Vinyl Ether Maleic-Anhydride Copolymer,” Journal of Chromatography, 1992, 597, 123-128.
“Immobilization of Saccharides and Peptides on 96-Well Microtiter Plates Coated with Methyl Vinyl Ether-Maleic Anhydride Copolymer,” Anal. Biochem., 1998, 260, 96-102.
“Surface Modification via Reactive Polymer Interlayers,” Langmuir 1996, 12, 2514-2518.
“Thin Polymer Layers as Supports for Hippocampal Cell Cultures,” Langmuir, 1998, 14, 3030-3035.
“Surface Esterification of Poly(Ethylene-alt-Maleic Anhydride) Copolymer,” J. Phys. Chem. B, 2000, 104, 10608-10611.
“Solventless Attachment of Long-Chain Molecules to Poly(ethylene-alt-maleic anhydride) Copolymer Surfaces,” J. Phys. Chem. B, 1998, 102, 5500-5502.
“Oligonucleotide Synthesis on Maleic Anhydride Copolymers Covalently Bound to Silica Spherical Support and Characterization of the Obtained Conjugates,” Journal of Applied Polymer Science, 1998, 70, 2487-2497.
“Oligonucleotide-Polymer Conjugates: Effect of the Method of Synthesis on Their Structure and Performance in Diagnostic Assays,” Bioconjugate Chemistry, 2000, 11, 795-804.
“Covalent Immobilization of Biological Molecules to Maleic Anhydride and Methyl Vinyl Ether Copolymers-A Physico-Chemical Approach,” Journal of Applied Polymer Science, 1999, 71, 927-936.
“Studies on Cyclic Anhydrides. IV. Rate Constants for the Hydrolysis of Some Cyclic Anhydrides Exhibiting Ring Strain,” Acta Chem. Scand., 1972, 26, 16-26.
“Covalent Immobilization of Proteins onto (Maleic Anhydride-alt-methyl Vinyl Ether) Copolymers: Enhanced Immobilization of Recombinant Proteins,” Bioconjugate Chemistry, 1998, 9, 655-661.
“Using Surface Plasmon Resonance to Study the Binding of Vancomycin and Its Dimer to Sel-Assembled Monolayers Presenting D-Ala-D-Ala,” J. Am. Chem. Soc. 1999, 121, 2629-2630.
“A Survey of Structure-Property Relationships of Surfaces that Resist the Adsorption of Protein,” Langmuir, 2001, 17, 5605-5620.
“Biosensor Analysis of the Interaction between Immobilized Human Serum Albumin and Drug Compounds for Prediction of Human Serum Albumin Binding Levels,” J. Med. Chem. 2000, 43, 1986-1992.
“Electrostatically Driven Immobilization of Peptides onto (Maleic Anhydride-alt-methyl Vinyl Ether) Copolymers in Aqueous Media,” Ladaviere et al., Bioconjugate Chem., 2000, 11, 1460-152.
“Reactive Polymers in Diagnostics: Synthesis and Characterizations of Nucleic Acid Probes and Maleic Anhydride-co-Methyl Vinyl Ether Polymers,” Ladaviere et al. J. Appl. Polymer Sci. 1997, 65, 2567-2577.
“Comparison of Methods for Immobilization to Enzyme-Linked Immunosorbent Assay Plates for the Detection of Sugar Chains,” Satoh et al., Anal. Biochem., 1999, 275, 231-235.
“Controlled Monomolecular Functionalization and Adhesion of Solid Surfaces,” Evenson et al., Chem Mater. 2000, 12, 3038-3043.
“Pellicular Immobilized Enzymes,” Horvath, Biochim. et Biophys. Acta, 1974, 358, 164-177.
“Immobilization of Proteins to a Carboxymethyldextran-Modified Gold Surface for Biospecific Interaction Analysis in Surface Plasmon Resonance Sensors,” Johnsson et al, Anal. Biochem., 1991, 198, 268-277.
“A Novel Hydrogel Matrix on Gold Surfaces in Surface Plasmon Resonance Sensors for Fast and Efficient Covalent Immobilization of Ligands,” Lofas and Johnsson, J. Chem. Soc. Chem. Commun., 1990, 1526-1528.
“Optimization of oligonucleotide-based DNA microarrays,” Relógio A, Schwager C, Richter A, Ansorge W, Valcárcel J., Nucleic Acids Res. 2002, 30, e51.
“Efficient transfer of large DNA fragments from agarose gels to diazobenzyloxymethyl-paper and rapid hybridization by using dextran sulfate,” Wahl G M, Stern M, and Stark G R, Proc Natl Acad Sci USA 1979, 76:3683-7.
“An evaluation of the performance of cDNA microarrays for detecting changes in global mRNA expression,” Yue H, Eastman P S, Wang B B, Minor J, Doctolero M H, Nuttall R L, Stack R, Becker J W, Montgomery J R, Vainer M, Johnston R, Nucleic Acids Res. 2001, 29, e41.
International Publication No. WO8706702
Japanese Publication No. JP 10234847
U.S. Pat. No. 4,407,975
U.S. Pat. No. 4,610,962
U.S. Pat. No. 4,610,962
U.S. Pat. No. 4,815,843
U.S. Pat. No. 6,541,071
U.S. Pat. No. 6,632,615
U.S. Publication No. US20030017464 A1
U.S. Publication No. US20040091602 A1
U.S. Publication No. US20040043497 A1
U.S. Publication No. US20040043508 A1
U.S. Pat. No. 5,436,161

What is claimed is:

1. A method for performing an assay of a sample comprising an analyte, the method comprising:
a. attaching a biomolecule to a support, wherein the support comprises a substrate, a first tie layer attached to the substrate, a first polymer attached to the first tie layer, wherein the biomolecule attaches to the first polymer;
b. exposing the support with the attached biomolecule to a relative humidity of greater than 40%;
c. attaching a blocking agent to the first polymer;
d. contacting support with the sample comprising the analyte, wherein after the contacting step, the analyte is bound to the biomolecule; and
e. detecting the bound analyte.

2. The method of claim 1, wherein the assay is a high-throughput assay.

3. The method of claim 1, wherein the analyte comprises a drug, an oligonucleotide, a nucleic acid, a protein, a peptide, an antibody, an antigen, a hapten, saccharide, lipid, a small molecule, or a mixture thereof.

4. The method of claim 1, wherein the bound analyte is detected by fluorescence or non-fluorescence.

5. The method of claim 1, wherein the bound analyte is detected by surface plasmon resonance, a waveguide resonant grating system, or mass spectrometry.

6. The method of claim 1, wherein the substrate comprises a plastic, a polymeric or co-polymeric substance, a ceramic, a glass, a metal, a crystalline material, a noble or semi-noble metal, a metallic or non-metallic oxide, a transition metal, or any combination thereof.

7. The method of claim 1, wherein the substrate comprises an amine modified glass surface.

8. The method of claim 1, wherein the substrate is a microplate or a slide.

9. The method of claim 1, wherein the first tie layer is covalently attached to the outer surface of the substrate.

10. The method of claim 1, wherein the first tie layer is electrostatically attached to the outer surface of the substrate.

11. The method of claim 1, wherein the first tie layer compound comprises a straight or branched-chain aminosilane, aminoalkoxysilane, aminoalkylsilane, aminoarylsilane, aminoaryloxysilane, or a derivative or salt thereof.

12. The method of claim 1, wherein the first tie layer compound comprises 3-aminopropyl trimethoxysilane, N-(beta-aminoethyl)-3-aminopropyl trimethoxysilane, N-(beta-aminoethyl)-3-aminopropyl triethoxysilane, N′-(beta-aminoethyl)-3-aminopropyl methoxysilane, or aminopropylsilsesquixoane.

13. The method of claim 1, wherein the first polymer is covalently attached to the first tie layer.

14. The method of claim 1, wherein the first polymer is electrostatically attached to the first tie layer.

15. The method of claim 1, wherein the first polymer comprises a copolymer.

16. The method of claim 15, wherein the copolymer is derived from maleic anhydride and a first monomer.

17. The method of claim 16, wherein the first monomer comprises styrene, tetradecene, octadecene, methyl vinyl ether, triethylene glycol methyl vinyl ether, butylvinyl ether, divinylbenzene, ethylene, acrylamide, pyrolidone, dimethylacrylamide, a polymerizable oligo(ethylene glycol) or oligo(ethylene oxide), or a combination thereof.

18. The method of claim 1, wherein the first polymer comprises poly(vinyl acetate-maleic anhydride), poly(ethylene-alt-maleic anhydride), poly(isobutylene-alt-maleic anhydride), poly(maleic anhydride-alt-1-octadecene), poly(maleic anhydride-alt-1-tetradecene), poly(maleic anhydride-alt-methyl vinyl ether), poly(triethyleneglycol methyvinyl ether-co-maleic anhydride), or any combination thereof.

19. The method of claim 1, wherein the first polymer comprises poly(styrene-co-maleic anhydride).

20. The method of claim 1, wherein the biomolecule comprises a natural, synthetic or modified oligonucleotide, a natural or modified nucleotide or nucleoside, a nucleic acid (DNA) or (RNA) or fragment thereof, a peptide comprising natural or modified amino acid, an antibody, a hapten, a biological ligand, a chelate, an aptamer, a lipid, a saccharide, a small molecule, a lectin, a modified polysaccharide, a synthetic composite macromolecule, a functionalized nanostructure, a synthetic polymer, a fluorophore, a chromophore, or a cell.

21. The method of claim 1, wherein the biomolecule comprises an oligonucleotide.

22. The method of claim 1, wherein the biomolecule comprises DNA or a fragment thereof.

23. The method of claim 1, wherein the biomolecule comprises RNA or a fragment thereof.

24. The method of claim 1, wherein the biomolecule comprises a protein, a peptide, or fragments thereof.

25. The method of claim 1, wherein the biomolecule is covalently attached to the first polymer.

26. The method of claim 1, wherein the biomolecule is electrostatically attached to the first polymer.

27. The method of claim 1, wherein the biomolecule is attached to the first polymer under a relative humidity of from about 40% to about 90%.

28. The method of claim 1, wherein the relative humidity is from about 55% to about 80%.

29. The method of claim 1, wherein the relative humidity is from about 60% to about 75%.

30. The method of claim 1, wherein during step (b), the substrate is exposed to a relative humidity of from 50% to 85% from 2 hours to 24 hours.

31. The method of claim 1, wherein during step (b), the substrate is exposed to a relative humidity of from 65% to 80% of from 8 hours to 16 hours.

32. The method of claim 1, wherein prior to step (a), the biomolecule is in a solution comprising a low boiling aprotic solvent, a surfactant, and a buffer, wherein the pH of the solution is from 7 to 10.

33. The method of claim 1, wherein a plurality of biomolecules are present on the support and wherein the biomolecules are on discrete and defined locations on the support to produce an array.

34. The method of claim 33, wherein the array comprises at least 96 distinct and defined locations.

35. The method of claim 33, wherein the support comprises at least 192 distinct and defined locations.

36. The method of claim 33, wherein the distinct and defined locations are from 50 to 1000 mm apart from each other.

37. The method of claim 1, wherein the blocking agent comprises at least one nucleophilic group, the first polymer comprises at least one electrophilic group, and the blocking agent is attached to the first polymer by a reaction between the electrophilic group and the nucleophilic group.

38. The method of claim 1, wherein the blocking agent comprises an amine group or a salt thereof.

39. The method of claim 1, wherein the blocking agent comprises 2-(2-aminoethoxy)ethanol, N,N-dimethyl ethylenediamine, ethanolamine, ethylenediamine, 4,7,10-trioxa-1,13-tridecanediamine, PEG amine, Tris hydrochloride, diethylaminoethyl-cellulose, diethylaminoethyl-cellulose amine, diethylaminoethyl-cellulose dextran, bovine serum albumin, chicken egg albumin, dry milk, pluronic or any combination thereof.

40. The method of claim 1, wherein the blocking agent comprises 2-(2-aminoethoxy)ethanol.

41. The method of claim 1, wherein the blocking agent comprises N,N-dimethyl ethylenediamine.

42. The method of claim 1, wherein the blocking agent comprises 2-(2-aminoethoxy)ethanol or N,N-dimethyl ethylenediamine in a solution of pH from 7 to 10.

43. The method of claim 1, wherein the blocking agent is covalently attached to the first polymer.

44. The method of claim 1, wherein the blocking agent is electrostatically attached to the first polymer.

45. The method of claim 1, wherein the sample of the analyte further comprises an enhancer.

46. The method of claim 45, wherein the enhancer comprises a low molecular weight poly(vinylpolypyrrolidone) polymer, low molecular weight dextran sulfate, Ficoll, low molecular weight polyethylene glycol, or any combination thereof.

47. The method of claim 45, wherein enhancer comprises poly(vinylpolypyrrolidone) having a molecular weight of about 10 kDa (PVP10), about 29 kDa (PVP29), about 50 kDa (PVP50), or about 55 kDa (PVP55), polyethyleneglycol having a molecular weight of about 8 kDa, or dextran sulfate having a molecular weight of about 8 kDa.

48. The method of claim 45, wherein the enhancer is present in the sample at from about 0.1% to about 30% by volume.

49. The method of claim 45, wherein the enhancer is present in the sample at from about 3% to about 4% by volume.

50. The method of claim 45, wherein the enhancer is present in the sample at from about 5% to about 10% by volume.

51. The method of claim 45, wherein the sample has a salt concentration of from 0.5×SSC to 20×SSC.

52. The method of claim 1, wherein the substrate is glass, the tie layer derived from gamma-aminopropylsilane, the first polymer is poly(styrene-co-maleic anhydride), the biomolecule is an oligonucleotide, wherein the relative humidity is from about 60% to about 75%, and the blocking agent is 2-(2-aminoethoxy)ethanol or N,N-dimethyl ethylenediamine.

53. A method for performing an assay of a sample comprising an analyte, the method comprising:
a. attaching a biomolecule to a support, wherein the support comprises a substrate comprising a first polymer, wherein the biomolecule attaches to the first polymer;
b. exposing the support with the attached biomolecule to a relative humidity of greater than 40%;
c. attaching a blocking agent to the first polymer;
d. contacting support with the sample comprising the analyte, wherein after the contacting step, the analyte is bound to the biomolecule; and
e. detecting the bound analyte.

54. The method of claim 53, wherein the substrate is derived from maleic anhydride and a first monomer.

55. The method of claim 54, wherein the first monomer comprises styrene, tetradecene, octadecene, methyl vinyl ether, triethylene glycol methyl vinyl ether, butylvinyl ether, divinylbenzene, ethylene, acrylamide, pyrolidone, dimethylacrylamide, a polymerizable oligo(ethylene glycol) or oligo(ethylene oxide), or a combination thereof.

56. The method of claim 53, wherein the first polymer comprises poly(vinyl acetate-maleic anhydride), poly(ethylene-alt-maleic anhydride), poly(isobutylene-alt-maleic anhydride), poly(maleic anhydride-alt-1-octadecene), poly(maleic anhydride-alt-1-tetradecene), poly(maleic anhydride-alt-methyl vinyl ether), poly(triethyleneglycol methyvinyl ether-co-maleic anhydride), or any combination thereof.

57. The method of claim 53, wherein the first polymer comprises poly(styrene-co-maleic anhydride).
